(19) United States
(12) Patent Application Publication
(10) Pub. No.: US 2026/0215954 A1
(43) Pub. Date: Jul. 30, 2026

(54) VERTICAL PATIENT POSITIONING AND IMMOBILIZATION BACKREST

(71) Applicant: MedTec LLC, Orange City, IA (US)

(72) Inventors: Brody De Jager, Alton, IA (US); Adam Howerzyl, Orange City, IA (US)

(21) Appl. No.: 19/148,542

(22) PCT Filed: Jan. 19, 2024
(86) PCT No.: PCT/US2024/012145

§ 371 Date: Jul. 16, 2025

Related U.S. Application Data
(60) Provisional application No. 63/481,694, filed on Jan. 26, 2023.

(51) Int. Cl. A61F 5/37

(52) U.S. Cl. CPC A61F 5/3769

(57) ABSTRACT

A vertical patient positioning device includes a backrest support comprising a first vertical planar surface. The vertical patient positioning device further includes a patient backrest comprising a second vertical planar surface, having mounting points for attaching at least one patient immobilization device, that attaches to the backrest support via a first and a second vertical adjustment assembly that enables the patient backrest to slide relative to the backrest support to adjust a vertical height of the patient backrest.

### FIELD OF THE INVENTION

The present invention is directed to a vertical backrest system for use in radiotherapy, diagnostic imaging, and other procedures involving positioning of a patient.

### BACKGROUND

Certain types of medical treatments or tests require that a portion of a human body be held in a same position to facilitate performance of the medical treatment or test upon that portion of the body. For example, different techniques have been used in the field of radiation oncology for supporting and holding body parts in a fixed position.

### SUMMARY OF THE INVENTION

According to one aspect, a vertical patient positioning device includes a backrest support comprising a first vertical planar surface. The vertical patient positioning device further includes a patient backrest comprising a second vertical planar surface, having mounting points for attaching at least one patient immobilization device, that attaches to the backrest support via a first and a second vertical adjustment assembly that enable the patient backrest to slide relative to the backrest support to adjust a vertical height of the patient backrest.

According to a further aspect, a backrest system for vertical patient positioning includes a backrest support configured to couple with a patient seat and comprising a first side surface and a second side surface. The backrest system further includes a backrest slidably attached to the first side surface of the backrest support via a first vertical adjustment assembly and to the second side surface of the backrest support via a second vertical adjustment assembly and further comprising a front surface, against which a back of a patient is positioned, wherein the front surface of the backrest includes a plurality of mounting or docking points for attaching at least one patient immobilization device. The first vertical adjustment assembly includes a first linear rail attached to the first side surface of the backrest support, and a first vertical adjustment mechanism that releasably engages with the first linear rail to adjust a vertical position of the backrest. The second vertical adjustment assembly includes a second linear rail attached to the second side surface of the backrest support, and a second vertical adjustment mechanism that releasably engages with the second linear rail to adjust the vertical position of the backrest.

According to an additional aspect, a vertical patient positioning device includes a backrest support comprising a first vertical planar surface, a patient backrest comprising a second vertical planar surface that includes multiple mounting or docking points for attaching at least one patient immobilization device, and at least one vertical adjustment assembly that interconnects the patient backrest with the backrest support to enable the patient backrest to slide vertically relative to the backrest support. The at least one vertical adjustment assembly includes a first linear rail that attaches to a first portion of the backrest support, a first dynamic rail that couples to the patient backrest, and a first actuation mechanism that causes, based on applied pressure, the first dynamic rail to either engage with the first linear rail to hold the patient backrest at a particular vertical height along the first linear rail relative to the backrest support or disengage from the first linear rail to enable the patient backrest to slide vertically relative to the backrest support. The backrest support may be configured to attach to a seat assembly.

### BRIEF DESCRIPTION OF THE DRAWINGS

FIG. 1 illustrates an exploded view of an example of a vertical backrest system for patient testing or treatment as described herein;

FIGS. 2A and 2B depict isometric views of the backrest system with a dynamic backrest being adjusted to its lowest vertical position relative to a backrest support of the backrest system;

FIGS. 3A and 3B depict the dynamic backrest of the backrest system being slid vertically relative to the backrest support;

FIGS. 4A and 4B illustrate close-up isometric views of the dynamic backrest being slid vertically relative to the backrest support;

FIGS. 5A and 5B are isometric views of an example of a vertical adjustment assembly of the backrest system;

FIG. 5C is an exploded view of an example of the vertical adjustment assembly of the backrest system;

FIGS. 6A-6C include different views of the release tab of the vertical adjustment mechanism;

FIGS. 7A and 7B depict a default state of the vertical adjustment assembly of the backrest system with a dynamic rail engaged with a linear static rail to hold the vertical adjustment mechanism in a particular vertical position;

FIGS. 8A and 8B depict a released state of the vertical adjustment assembly, with the dynamic rail disengaged from the linear static rail to enable the vertical adjustment mechanism to move vertically to adjust the vertical position of the dynamic backrest of the backrest system;

FIG. 9 illustrates an example of the mounting of the backrest system described herein to an underlying seat assembly such that the backrest system may be used for the vertical positioning and immobilization of a patient for treatment or testing;

FIG. 10 illustrates one example of a reference system for use in setting a position of the dynamic backrest relative to the backrest support of the backrest system;

FIG. 11 illustrates an example of incremental visual markings on a front surface of the dynamic backrest that may be used for affixing various devices or accessories to the backrest at particular positions;

FIG. 12 illustrates an example of a device that may be slidably attached to the dynamic backrest of the backrest system; and

FIG. 13 illustrates a method of use of the vertical backrest system described herein.

### DETAILED DESCRIPTION OF THE PREFERRED EMBODIMENTS

The following detailed description refers to the accompanying drawings. The same reference numbers in different drawings may identify the same or similar elements. The following detailed description does not limit the invention.

A technique, in the medical treatment/testing fields, for positioning and holding body parts in a fixed position uses heat-formable structures that include a sheet of retention material that is stretched over the body part(s) of the patient. For example, for performing radiation treatment of the head, thorax, breast, abdomen, hip, or pelvic region of a patient, the heat-formable structure includes a patient positioning mold having a sheet of retention material that is stretched over the patient's body part (e.g., head, breast, abdomen, hip, pelvic region). To form the mold over the patient's body part, a hot water bath or oven may be first used to heat the material of the heat-formable structure such that the sheet of material becomes pliable and deformable. The heat-formable sheet is then stretched over the patient's body part, and the sheet is allowed to cool and harden, permanently forming the sheet to the shape of the body part of the patient. As an example, a mold having a sheet of thermoplastic retention material, after heating, may be stretched over a patient's face and head, and then allowed to cool. Upon cooling, the mold, formed to the patient's face and head, creates a structure that can be used to hold the patient's head in a fixed position during, for example, radiation treatments. After the sheet of thermoplastic retention material of the mold is stretched over the body part of the patient, a frame portion of the mold is attached to a patient support table, a base, or other structure, using an attachment or docking mechanism(s).

A wide variety of existing systems for immobilizing patient body portions or parts are available for sale across the world. These existing systems typically include means for positioning a patient for radiation treatment of cancer (e.g., using photons, electrons, protons, or other heavy ions), or for medical imaging (e.g., Magnetic Resonance Imaging (MRI), Computed Tomography (CT), among others), of, for example, the head, thorax, breast, abdomen, hip, or pelvic regions of patients. Exemplary systems often include some form of a horizontal table or horizontal overlay upon a table or couchtop structure that uses various immobilization devices, such as thermoplastic docking devices. Treatment or testing of a patient in a vertical position, however, as opposed to existing horizontal systems, enables the patient's body to be oriented in a more natural and comfortable position during the treatment or testing. Vertical positioning of patients further allows the facility performing the treating or testing to increase patient throughput due to the natural orientation of the patients and the ease and speed with which the patients can be positioned and immobilized in the vertical position.

Vertical positioning systems, however, pose additional challenges. A main challenge is the accommodation of a variety of patient anatomy sizes. This may require the use of separate supports configured to generically support a range of patient sizes, such as pediatric or adult patients, which can pose a logistical challenge for storage in the clinic. Additionally, such vertical patient supports may not be suitable for all imaging or treatment modalities. For example, the construction of such supports may create undesirable imaging artifacts or undesirable attenuation or inhomogeneities for treatment applications such as radiotherapy. An objective problem to be solved is that of creating an adaptable vertical positioning system that can accommodate various sizes of patient anatomy while maintaining desirable characteristics for diagnostic imaging and radiotherapy applications.

The present invention describes a vertical patient positioning and immobilization backrest system that enables treatment and testing of a patient in a substantially vertical position while accommodating a wide variety of patient anatomies. The vertical positioning and immobilization backrest system includes vertical adjustment assemblies that each may include a spring loaded, toothed dual rail system which allows for the vertical adjustment of varying patient heights in a very quick, repeatable, and efficient manner. The vertical adjustment assemblies may reside at each side of the backrest system and couple a backrest support of the backrest system to a dynamic backrest that slides adjacent to, and vertically relative to, the backrest support. The toothed dual rail system includes a linear static rail that attaches to each side surface of the backrest support, and a dynamic rail having teeth that releasably engage with teeth of the static rail to “lock” the dynamic rail, and the dynamic backrest that is attached to the dynamic rail, at a particular vertical height relative to the backrest support. Use of the spring loaded, toothed dual rail system enables efficient and repeatable adjustment of a height of the dynamic backrest for vertical positioning and immobilization of the patient upon the dynamic backrest.

The vertical patient positioning and immobilization backrest system further includes at least one reference system that may be used to index the patient's position in a repeatable manner. The reference system may include at least one indicator that indicates the position of the backrest relative to the rail system. The at least one reference system may include one or more of the following: scales or other incremental visual markings, tactile or haptic feedback, a clicking sound or other auditory feedback, visual indicators such as a needle or marker related to a series of visual markings, and/or other types of references or indicators known to a person having ordinary skill in the art. In one embodiment, the at least one reference system may be disposed on the system to enable repeatable adjustment to a particular height setting on the rail system. In another embodiment, the at least one reference system may enable indexing of accessories, such as cushions and attachable positioners in the superior to inferior direction. In a further embodiment, the at least one reference system may be disposed on at least one side of the device. In an additional embodiment, the at least one reference system enables the attachment of accessories at predefined discrete positions by an attachment means such as a locating bar. In yet another embodiment, the at least one reference system may indicate a particular angle of adjustment of the backrest. As would be understood by one having ordinary skill in the art, multiple types of reference systems and corresponding indicators may be provided to enable indexing of various aspects of the height and/or angle of the at least one backrest, the patient's position, and/or any accessories required to position the patient.

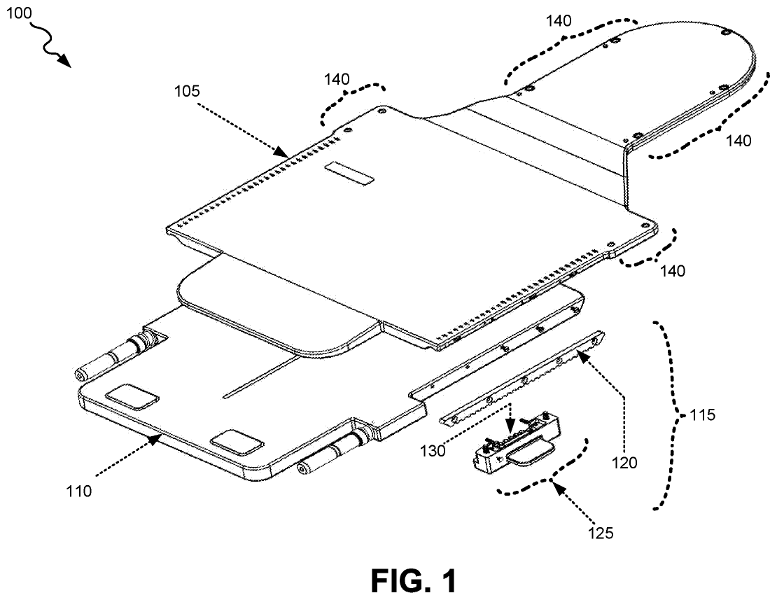

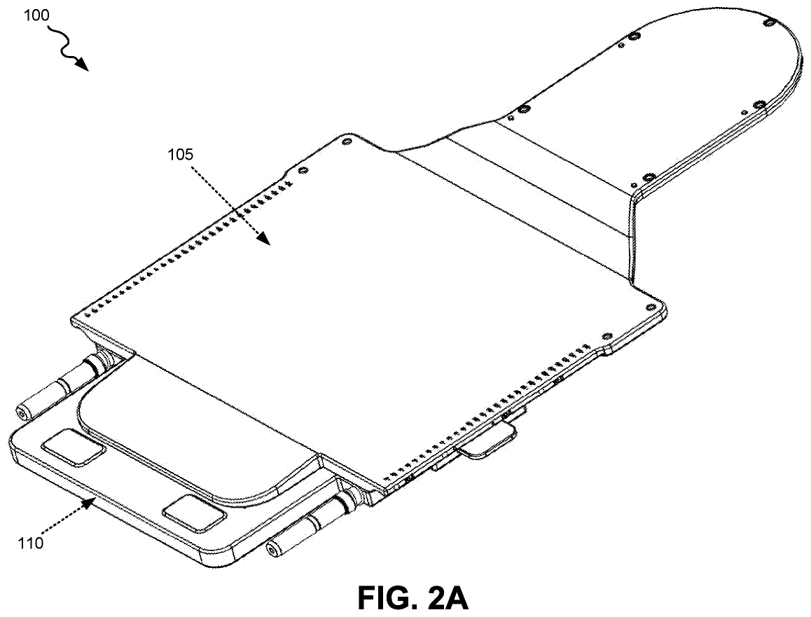

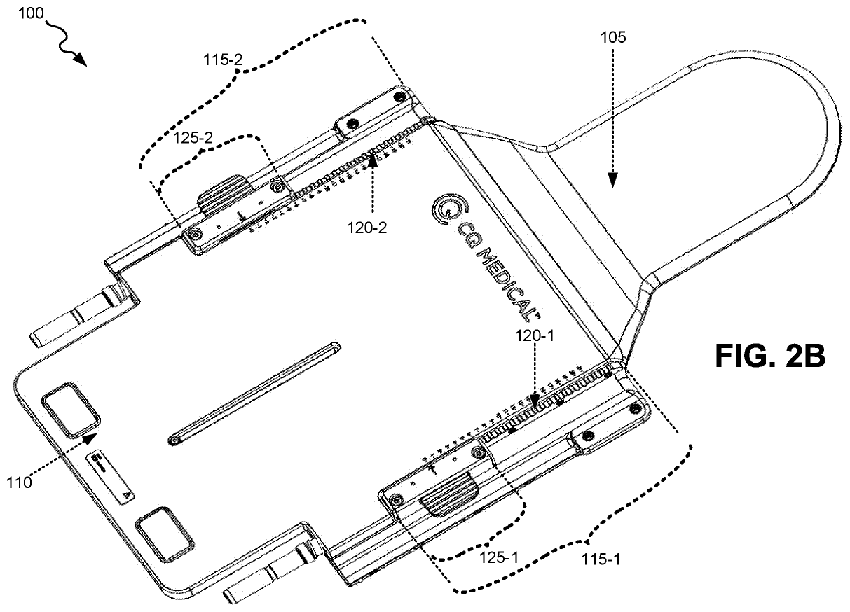

Referring to FIG. 1, the backrest system 100 (also referred to herein as “backrest device 100”) includes a dynamic backrest 105, at least one backrest support 110, and at least one vertical adjustment assembly 115. The backrest system 100 is shown in a horizontal position for purposes of illustration, whereas actual use of the backrest system 100 involves the backrest system 100 being in a vertically oriented position, or a substantially vertically oriented position. Only a single vertical adjustment assembly 115 is shown in the embodiment depicted in FIG. 1. The embodiment of FIG. 1 may, in some implementations, include at least one additional vertical adjustment assembly 115 that is not shown (e.g., located on a side of system 100 opposite to that of the vertical adjustment assembly 115 in FIG. 1, such as shown in FIG. 2B). As shown, the dynamic backrest 105 couples to the at least one backrest support 110. In one embodiment, this coupling is accomplished via the at least one vertical adjustment assembly 115. In another embodiment, this coupling is accomplished via multiple vertical adjustment assemblies 115. The at least one vertical adjustment assembly 115, or multiple vertical adjustment assemblies 115, may be positioned laterally relative to a vertically oriented center axis of the at least one backrest support 110 and backrest 105. In one embodiment, the at least one vertical adjustment assembly 115, or multiple vertical adjustment assemblies 115, may be positioned at a side surface of the at least one backrest support 110 (only a single vertical adjustment assembly 115 at a single side surface is shown in FIG. 1). In some embodiments (not shown), the backrest system 100 may be capable of angular adjustment for patient comfort and additional flexibility in patient positioning and to enable different imaging or treatment angles. For example, in one embodiment, angulation of backrest system 100 may be provided for 15 degrees from a neutral vertical direction in the forward and backward directions, facilitating a total adjustment range of 30 degrees

A back surface of dynamic backrest 105, as shown in FIG. 1, may be located adjacent to a front surface of backrest support 110. Dynamic backrest 105 may include a planar member having a shape that approximates an outline shape of the body part or parts of the patient which is/are to be immobilized against dynamic backrest 105. In the example of FIG. 1, dynamic backrest 105 includes a planar member that approximates the shape of the head, shoulders, and torso of a patient. Dynamic backrest 105 has two lateral edges to which respective vertical adjustment assemblies 115 may be coupled. The at least one dynamic backrest 105 may be configured such that the area around the vertically oriented center axis, corresponding to the patient's spinal column and the clinical region of interest, is radiolucent, homogeneous to x-rays, low attenuating, and substantially free from obstructions or inhomogeneities. The at least one backrest support 110 may be configured such that imaging or treatment is capable of being delivered in a substantially unobstructed manner through the dynamic backrest. The at least one backrest support 110 may include a planar member having a roughly rectangular shape and having two side surfaces, with each of the two side surfaces being coupled to a respective vertical adjustment assembly 115. The at least one backrest support 110 may itself be homogeneously constructed and substantially free from obstructions or inhomogeneities in a clinical region of interest. In particular, the homogeneous construction of the at least one backrest support 110 may be constructed in a manner such that it has consistent attenuation that can be factored into the imaging parameters or treatment plan as may be deemed clinically appropriate. In one implementation, the planar members of dynamic backrest 105 and/or backrest support 110 may be made from a strong and resilient material such as, for example, carbon fiber or other fiber reinforced composite material. Other types of materials may, however, be alternatively used, such as different types of polymer materials or metals capable of supporting an immobilized patient. In one embodiment, the planar members of the dynamic backrest 105 and/or the at least one backrest support 110 may be made from materials that are radiolucent and homogeneous to x-rays. In another embodiment, the planar members of the dynamic backrest 105 and/or the at least one backrest support 110 may be made from materials that are capable of being utilized in an MR environment, particularly materials that are non-magnetic, non-conductive, and non-metallic.

The at least one vertical adjustment assembly 115 includes a linear static rail 120 and a vertical adjustment mechanism 125 that includes, among other components, a dynamic rail 130. As described further below, teeth of the dynamic rail 130 releasably engage with teeth of the linear static rail 120 to “lock” the dynamic backrest 105 in a particular position relative to the backrest support 110 or to “unlock” the dynamic backrest 105 and enable backrest 105 to be slid vertically (e.g., up or down) relative to backrest support 110. Therefore, disengagement of the teeth of the dynamic rail 130 from the teeth of the linear static rail 120 enables dynamic backrest 105 to be slid vertically relative to backrest support 110, and engagement of the teeth of the dynamic rail 130 with the teeth of the linear static rail 120 holds the vertical position of dynamic backrest 105 relative to backrest support 110. The various different components of the vertical adjustment mechanism 125, and their operation, are described further below.

As further shown in FIG. 1, a front surface of dynamic backrest 105 may include multiple mounting or docking points 140 for attaching at least one patient immobilization device, or other structure(s) or mechanism(s) (not shown in FIG. 1). The configuration, and type, of the multiple mounting or docking points 140 may vary depending on the patient immobilization device(s) that are to be attached to dynamic backrest 105 to immobilize the patient against backrest 105. As one example, a thermoplastic mask (not shown) may be docked to a particular arrangement of docking points on an upper portion of backrest 105 to immobilize a patient's head upon backrest 105. The upper portion of backrest 105 may have a different shape, with a different configuration and/or type of mounting or docking points, than that shown in FIG. 1. Dynamic backrest 105 may be configured with multiple mounting or docking points 140 for attaching one or more of numerous different types, and designs, of devices, structures, or mechanisms, such as, for example, patient immobilization devices.

As described further below, a lower portion of backrest support 110 and/or dynamic backrest 105 may be attached to an underlying seat assembly (not shown in FIG. 1) that enables a patient to sit in an upright, vertical position with their back (or chest and abdomen) resting against backrest 105.

FIGS. 2A and 2B depict isometric views of backrest system 100 with the dynamic backrest 105 adjusted to its lowest vertical position relative to backrest support 110. FIG. 2A illustrates a front isometric view of backrest system 100, and FIG. 2B illustrates a rear isometric view of backrest system 100. As shown in FIG. 2A, the substantially flat, planar member of dynamic backrest 105 resides adjacent to backrest support 110 such that dynamic backrest 105 may slide vertically (e.g., parallel to a primary plane of backrest support 110), via use of vertical adjustment assembly 115 (not fully shown in FIG. 2A), without obstruction by backrest support 110. In the rear isometric view of FIG. 2B, a first linear static rail 120-1 attaches to a first side surface of the backrest support 110, and a second linear static rail 120-2 attaches to a second side surface of the backrest support 110. Each vertical adjustment mechanism 125-1 and 125-2 attaches to a rear surface of backrest 105 such that backrest 105 can be slid vertically relative to backrest support 110 upon linear static rails 120-1 and 120-2.

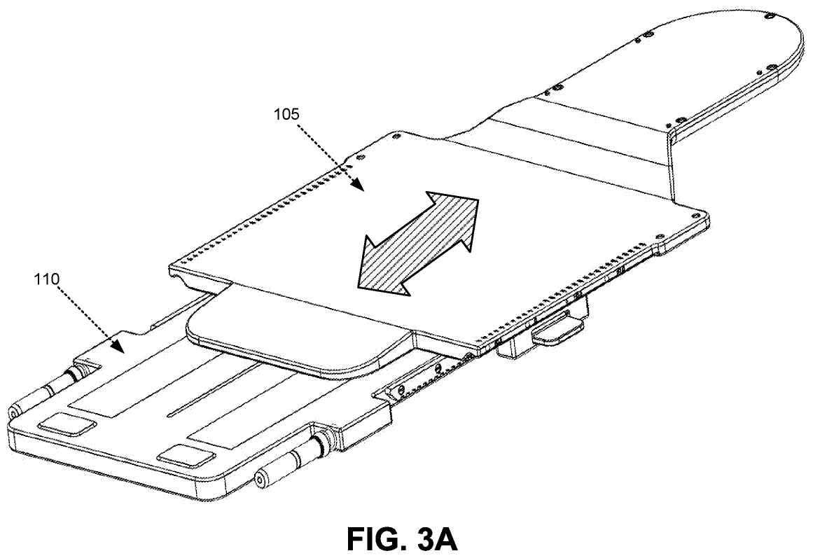

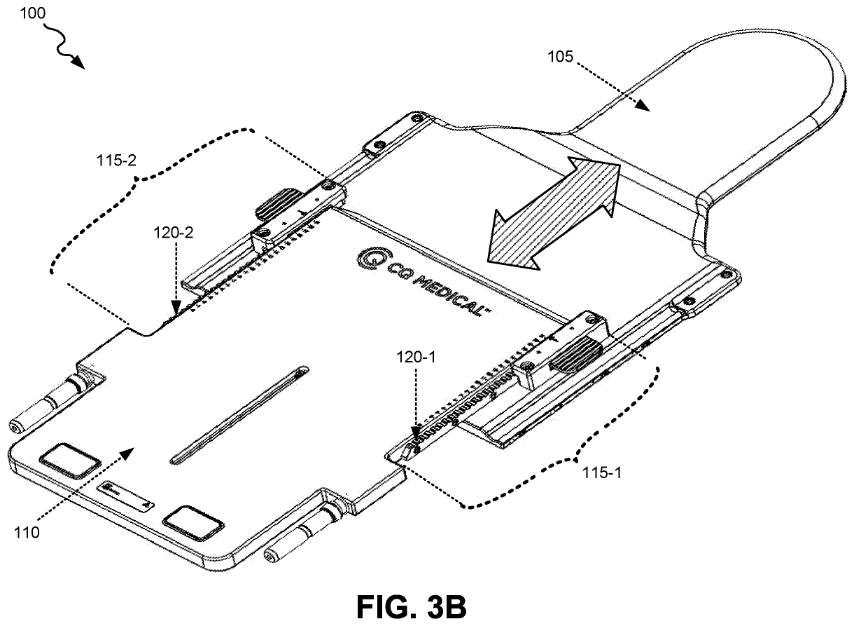

FIGS. 3A and 3B depict an example of dynamic backrest 105 being slid vertically relative to backrest support 110. In the illustrations of FIGS. 3A and 3B, upwards movement of dynamic backrest 105 is towards the upper right corner, and downwards movement of dynamic backrest 105 is towards the lower left corner. In the example of FIGS. 3A and 3B, dynamic backrest 105 has been slid to the upper extent of permitted vertical movement. Dynamic backrest 105 may be slid (not shown in FIGS. 3A and 3B) to return backrest 105 to the lowest extent of permitted movement, or to various intermediate positions.

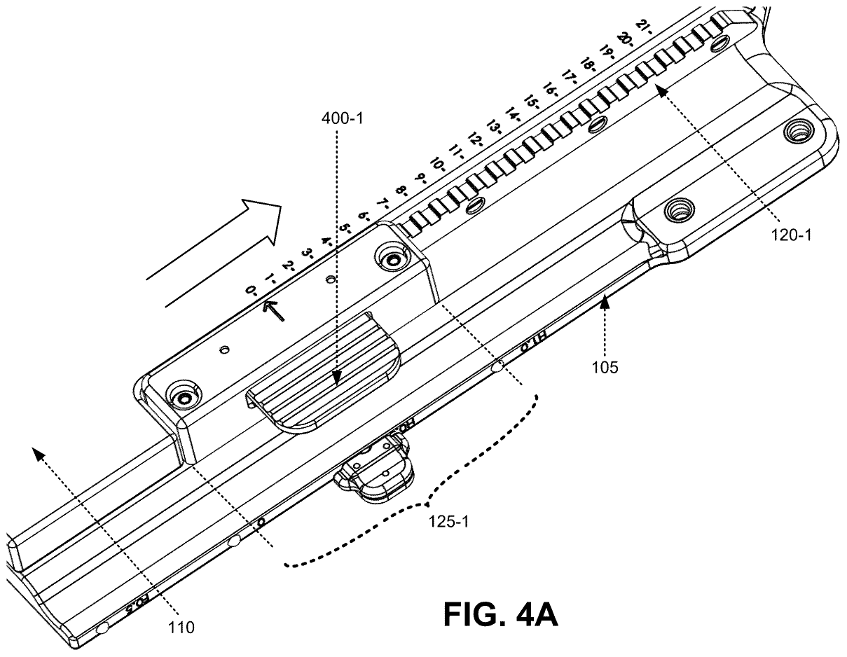

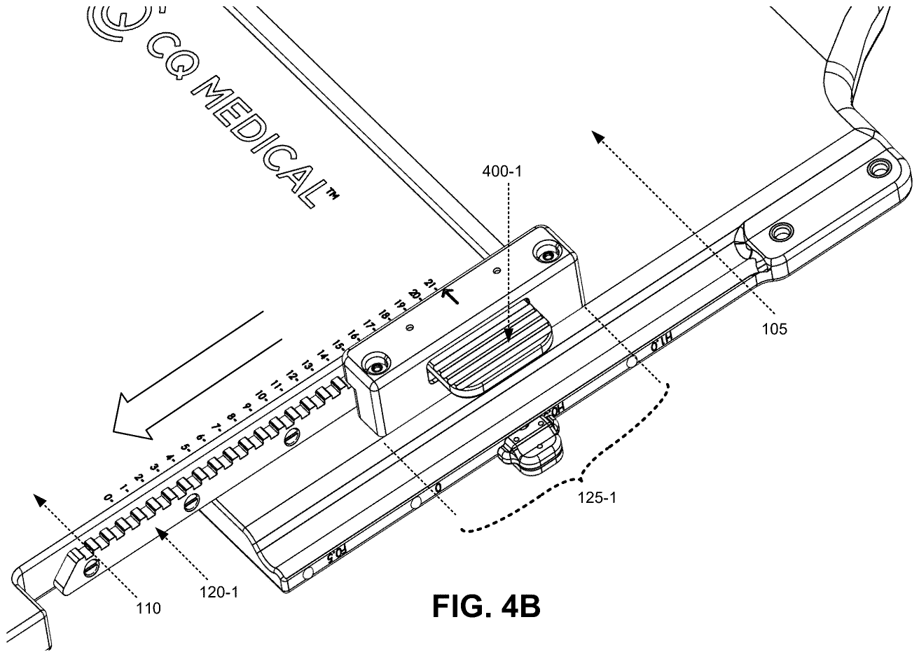

FIGS. 4A and 4B illustrate close-up isometric views of dynamic backrest 105 being slid vertically relative to backrest support 110. FIG. 4A shows vertical adjustment mechanism 125-1 starting at its lowest extent of vertical adjustment upon linear static rail 120-1. Upon pressing of release tab 400-1 of vertical adjustment mechanism 125-1 (and a simultaneous pressing of a release tab 400-2 on vertical adjustment mechanism 125-2—not shown in FIG. 4A), dynamic backrest 105 may be slid upwards (i.e., towards the upper right corner of FIG. 4A) relative to backrest support 110 by the application of upwards force upon dynamic backrest 105 (e.g., applied by a system operator) to various intermediate positions along linear static rail 120-1, or to a highest extent of vertical adjustment. A spacing interval of the teeth of linear static rails 120 and dynamic rails 130 determines a gradation of height adjustments that may be made by sliding vertical adjustment mechanisms 125 along linear static rails 120. For example, smaller spacing intervals between the teeth of the linear static rails 120 and dynamic rails 130 permits a “finer” gradation of height adjustments, whereas larger spacing intervals between the teeth of the linear static rails 120 and dynamic rails 130 results in a “coarser” gradation of height adjustments.

FIG. 4B shows vertical adjustment mechanism 125-1 reaching its highest extent of vertical adjustment upon linear static rail 120-1. Upon pressing (downwards in the view of FIG. 4B) of release tab 400-1 of vertical adjustment mechanism 125-1 (and a simultaneous pressing of a release tab 400-2 on vertical adjustment mechanism 125-2—not shown in FIG. 4B), dynamic backrest 105 may be slid downwards (i.e., towards the lower left corner of FIG. 4B) relative to backrest support 110 by the application of a downwards force upon dynamic backrest 105.

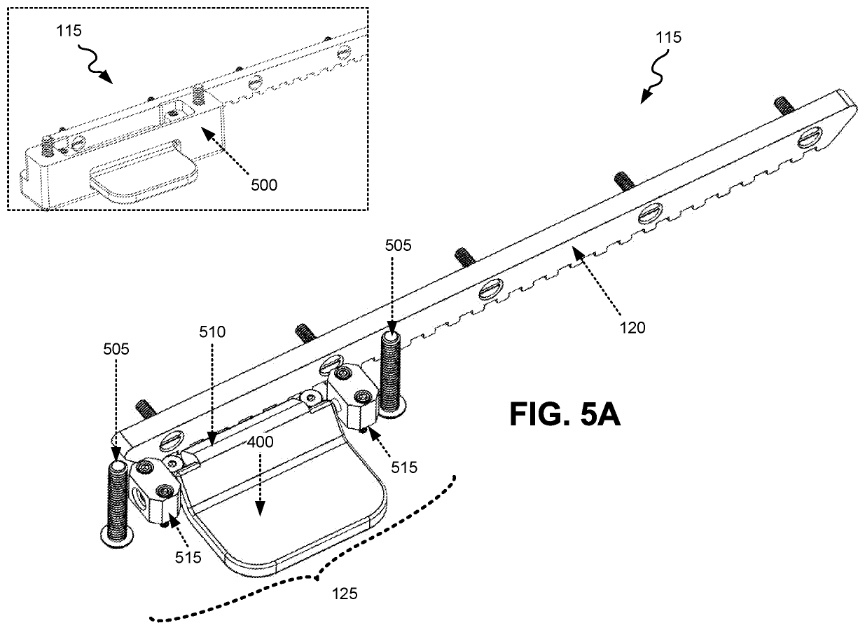

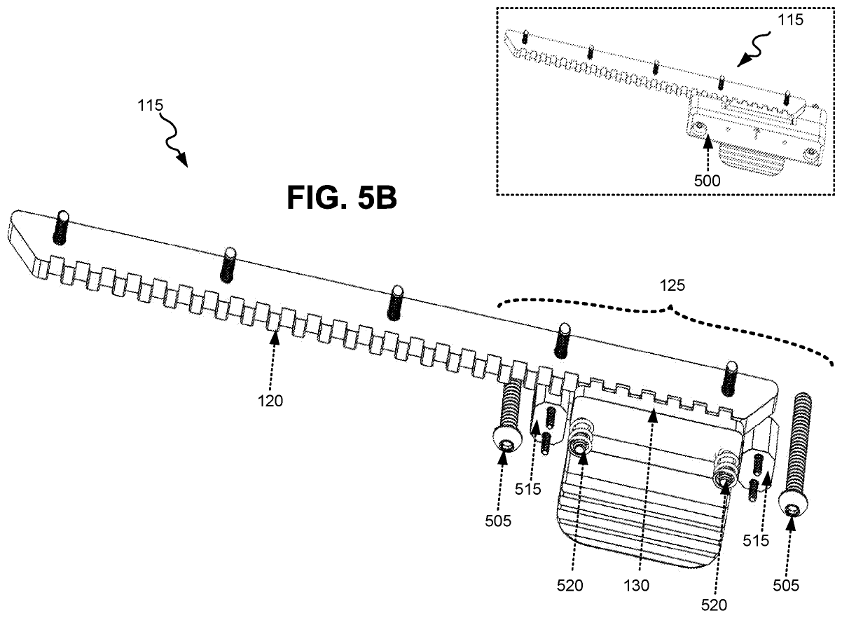

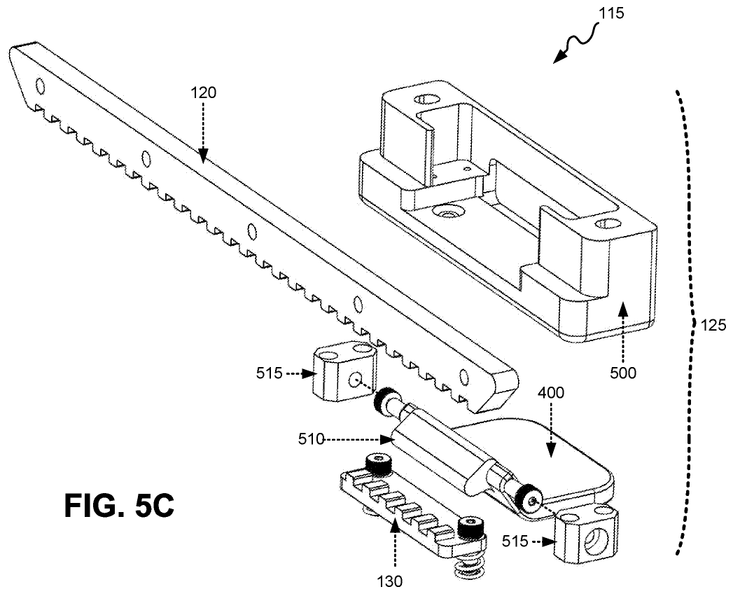

FIGS. 5A and 5B are isometric views, and FIG. 5C is an exploded view, of one example implementation of vertical adjustment assembly 115. In the larger isometric views of FIGS. 5A and 5B, vertical adjustment assembly 115 is shown with an invisible release tab housing 500 to enable views of internal components of vertical adjustment mechanism 125. As shown in the example of FIGS. 5A, 5B, and 5C, vertical adjustment assembly 115 includes a vertical adjustment mechanism 125 and a linear static rail 120. Vertical adjustment mechanism 125 further may include a number of components, including release tab 400, release tab housing 500, housing mounting screws 505, a release actuator 510, pivot blocks 515, compression springs 520 (FIG. 5B), and a dynamic rail 130 (FIG. 5C). Housing mounting screws 505 screw into a rear surface of dynamic backrest 105 (not shown) to mount release tab housing 500 to backrest 105 against linear static rail 120.

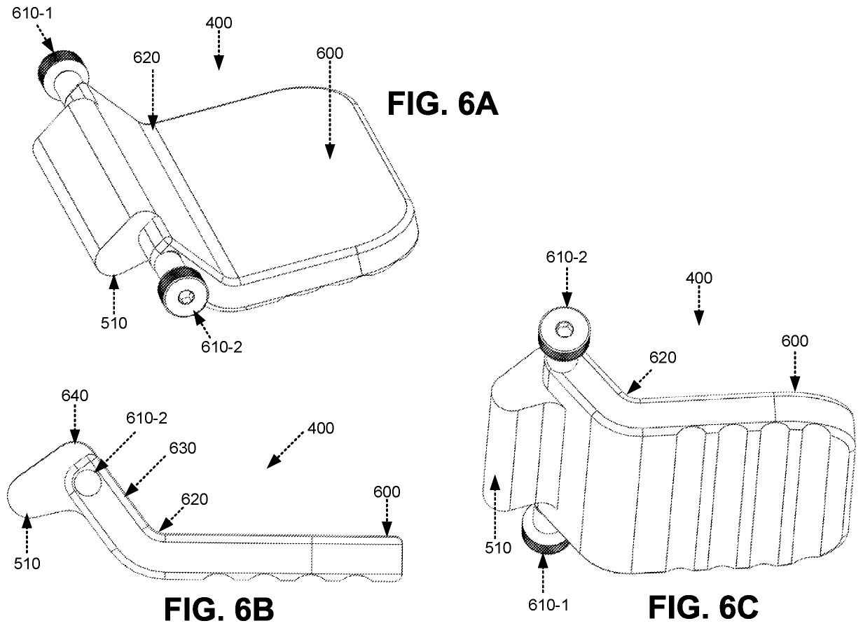

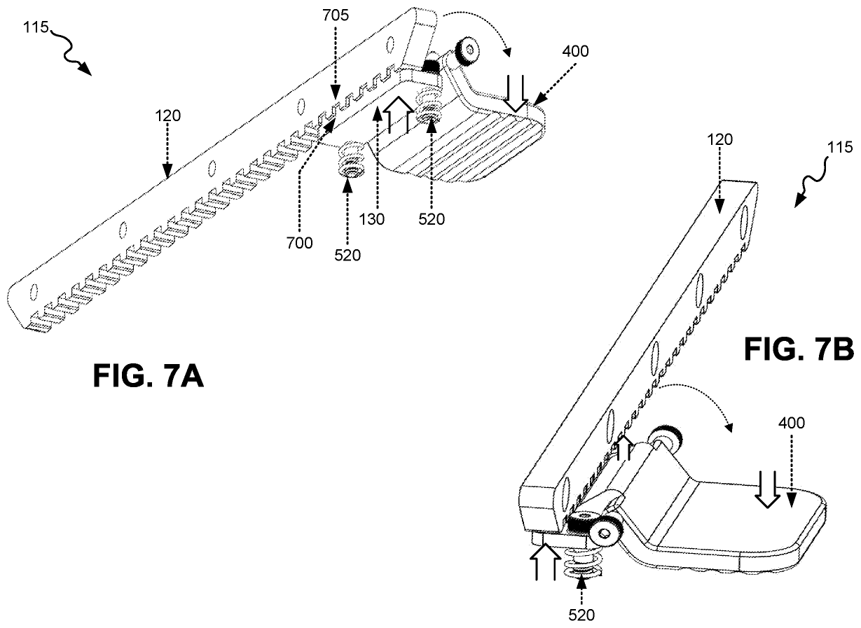

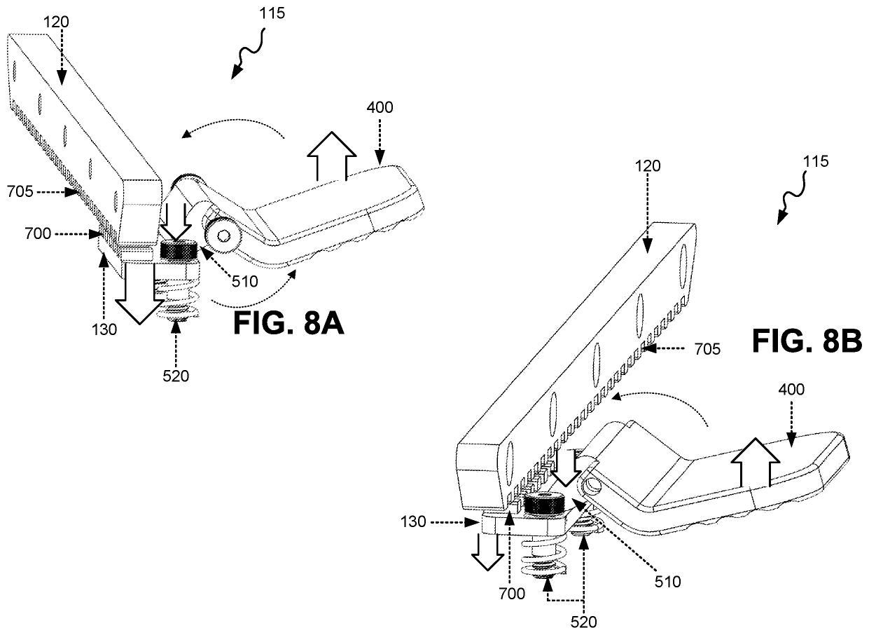

Release tab 400 includes a lever-like structure having a pivot axis that pivots within the pivot blocks 515 based on pressure applied to release tab 400. FIGS. 6A-6C include different views that show the various components of release tab 400. As shown, release tab 400 includes a paddle arm 600, pivot arms 610-1 and 610-2, and release actuator 510. Release tab 400 may include a unitary device made from a single material (e.g., plastic), or may be composed from multiple components connected to, or inserted into, one another. In the latter implementation, for example, pivot arms 610-1 and 610-2 may be separate components that are inserted into holes within the outer edges of release tab 400. Paddle arm 600 includes a roughly rectangular and planar member having a surface to which force may be applied to cause the lever-like structure of release tab 400 to pivot, via operation of pivot arms 610-1 and 610-2, causing the release actuator 510, in lever-like fashion, to apply force against a flange of dynamic rail 130 (as described further below). Release tab 400 may have a roughly “arm and shoulder” configuration in which the paddle arm 600 connects, at an elbow region 620, to an upper arm 630 (e.g., at an approximate 60 degree angle between paddle arm 600 and upper arm 630), which, in turn, connects to a shoulder region 640. Shoulder region 640 of release tab 400 further connects to release actuator 510. Further details of the components, and operation, of the vertical adjustment mechanism assembly 115 are described below with respect to FIGS. 7A, 7B, 8A, and 8B.

In some implementations, the release tab housing 500 of the vertical adjustment mechanism 125 may be a self-contained unit, which can be uncoupled without internal components becoming dislodged or disassembled. This not only facilitates servicing or replacement of the backrest system 100, the at least one vertical adjustment assembly 115, or other constituent parts of the unit, but also enables the backrest to be interchangeable, among multiple different types of backrests, to suit various clinical indications as may be needed for effective treatment and patient positioning. In further implementations, the release tab housing 500 may include an opening to enable access to a release button (not shown) associated with the dynamic rail 130. This enables a user, without uncoupling the release tab housing 500 from the dynamic backrest 105, to engage the release button and detach the dynamic backrest 105, including vertical adjustment mechanism 125, from backrest support 110.

FIGS. 7A and 7B are two different isometric views of portions of vertical adjustment assembly 115, with release tab housing 500 and pivot blocks 515 rendered invisible to enable a clear view of the operation of the internal components of vertical adjustment mechanism 125. FIGS. 7A and 7B depict a default state of vertical adjustment assembly 115, with the compression force of compression springs 520 causing dynamic rail 130 to engage with linear static rail 120 to hold vertical adjustment mechanism 125 (and dynamic backrest 105 to which the mechanism 125 is attached) in a particular vertical position relative to backrest support 110. Orientation terms (e.g., upwards, downwards, upper, lower), applied to the disposition of, and/or the operation of, the components of vertical adjustment assembly 115 in the description below, refer to orientations in the context of the particular pictorial representations, and not to actual physical orientations when system 100 is in a vertical, upright position during positioning and immobilization of a patient.

As shown in FIGS. 7A and 7B, compression springs 520 apply upwards force to a lower surface of a flange of dynamic rail 130, moving dynamic rail 130 against linear static rail 120 (“upwards” in the depiction of FIGS. 7A and 7B) such that the teeth 700 of dynamic rail 130 engage with the teeth 705 of linear static rail 120. Upwards movement of the flange of dynamic rail 130 applies force to release actuator 510, causing the lever-like structure of release tab 400 to rotate (clockwise in the example of FIGS. 7A and 7B), pushing down release tab 400 to its lowest vertical position (indicating an unreleased state of vertical adjustment mechanism 125).

FIGS. 8A and 8B are also two different isometric views of vertical adjustment assembly 115, with release tab housing 500 and pivot blocks 515 rendered invisible to enable a clear view of the internal components of vertical adjustment mechanism 125. FIGS. 8A and 8B depict a released state of vertical adjustment assembly 115, with an applied force to release tab 400 (e.g., by a system operator) causing dynamic rail 130 to disengage from linear static rail 120 to enable vertical adjustment mechanism 125 to move vertically along linear static rail 120 to adjust the vertical position of dynamic backrest 105.

As shown in FIGS. 8A and 8B, when release tab 400 is depressed (“upwards” in the depiction of FIGS. 8A and 8B) by a system operator (not shown), release tab 400 moves in a lever-like fashion, causing release actuator 510 to push downwards (shown as counter-clockwise in the example of FIGS. 8A and 8B) against an upper surface of the flange of the dynamic rail 130. The downwards force against the upper surface of the flange of dynamic rail 130 causes dynamic rail 130 to move downwards, against the opposing upwards force of compression springs 520, such that the teeth 700 of dynamic rail 130 disengage from the overlying teeth 705 of linear static rail 120. Disengagement of dynamic rail 130 from the linear static rail 120 enables vertical adjustment mechanism 125, through application of an upwards or downwards force (e.g., by the system operator) to vertical adjustment mechanism 125 and/or dynamic backrest 105, to be slid vertically along the track of linear static rail 120. Once the pressure upon release tab 400 is discontinued (i.e., the system operator stops pressing release tab 400), the force of compression springs 520 causes the teeth of the dynamic rail 130 to re-engage with the teeth of the linear static rail 120, as described above with respect to FIGS. 7A and 7B.

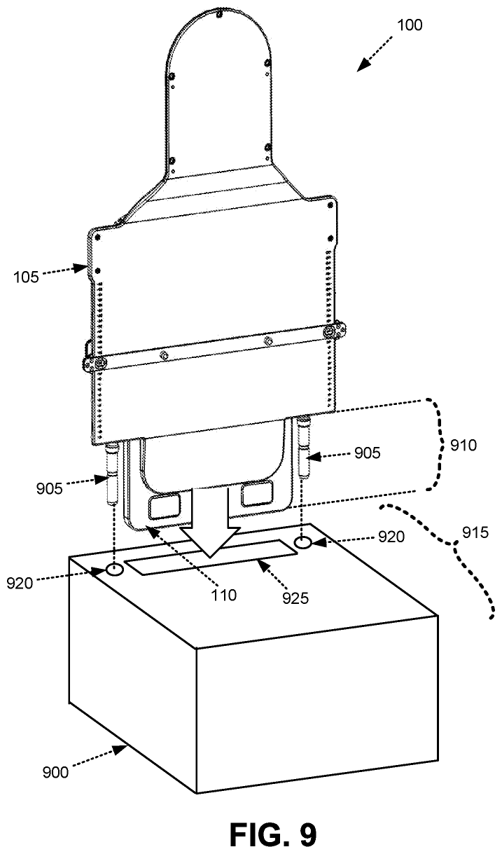

FIG. 9 illustrates an example of the mounting or attachment of backrest system 100 to an underlying seat assembly 900 such that backrest system 100 may be used for the vertical positioning and immobilization of a patient for treatment or testing. The example of FIG. 9 depicts seat assembly 900 as including a generic cube-like structure having a corresponding mounting slot and guide holes for mounting the backrest system 100. Seat assembly 900, however, may include various different designs, having different configurations, that include different types of mounting points and/or mounting mechanisms for mounting backrest system 100 to the seat assembly 900, that are not illustrated in FIG. 9.

As shown in FIG. 9, backrest system 100 may further include two guide rods 905, located on each side of a lower surface of backrest support 110, and an insertion portion 910 of backrest system 100, that insert into corresponding holes and slots in an upper surface 915 of seat assembly 900. For example, with backrest system 100 in a vertically oriented position as shown, guide rods 905 may be inserted into guide rod holes 920 within the upper surface 915 of seat assembly 900. Additionally, insertion portion 910 of backrest system 100 may simultaneously be inserted into a retention slot 925 in the upper surface 915 of seat assembly 900. Guide rod holes 920 and retention slot 925 hold backrest system 100 in its vertically oriented position relative to seat assembly 900 such that a patient may sit upon the seat (e.g., the upper surface 915 of seat assembly 900) to be positioned and immobilized. In the example of FIG. 9, backrest system 100 is held by seat assembly 900 in a vertically oriented, free-standing position, without requiring backrest system 100 to be connected to any other support structure (e.g., a wall).

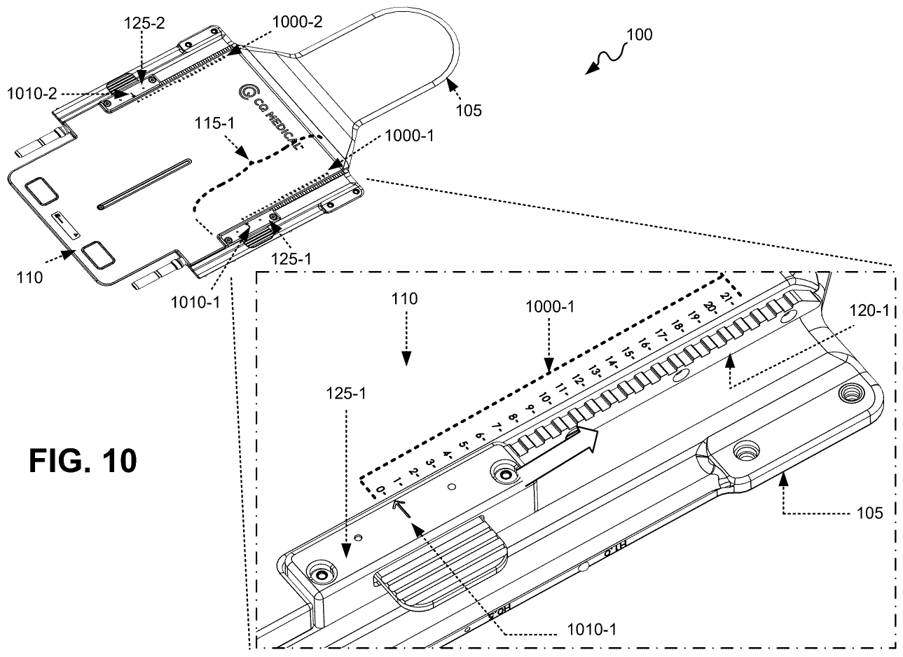

FIG. 10 depicts one example of a reference system for use in setting a position of the dynamic backrest 105 relative to the backrest support 110 of backrest system 100. In FIG. 10, backrest system 100 is viewed from behind backrest support 110, with the back side of dynamic backrest 105 (i.e, the side opposite which the patient is seated against backrest 105) being visible. As shown, a portion of the reference system includes incremental visual markings 1000-1 located upon the outer surface of the back side of backrest support 110 and aligned parallel to linear static rail 120-1. The incremental visual markings 1000-1 may include evenly spaced reference markings that provide an indication of the adjusted height of the dynamic backrest 105 relative to the backrest support 110. The reference markings may, for example, be spaced ⅛, ¼, or ⅜ inch apart, and include a sequence of numbers (e.g., integer numbers 0-21 shown in FIG. 10) that enable the operator to repeatably adjust dynamic backrest 105 to a precise height. The portion of the reference system further includes an adjustment indicator line 1010-1, located upon an outer surface of vertical adjustment mechanism 125-1, that extends perpendicular to linear static rail 120-1 and the incremental visual markings 1000-1. As vertical adjustment mechanism 125-1 is used to raise (shown with an arrow in the lower right corner of FIG. 10) or lower dynamic backrest 105 relative to backrest support 110, the operator (not shown) may visually align adjustment indicator line 1010-1 with a particular reference marking of the incremental visual markings 1000-1 to adjustment dynamic backrest 105 to a precise height relative to backrest support 110.

Though not shown in detail in FIG. 10, another portion of the reference system includes identical incremental visual markings 1000-2 located upon the outer surface of the the back side of backrest support 110 and aligned parallel to linear static rail 120-2. These incremental visual markings 1000-2 may also be used, in conjunction with an adjustment indicator line 1010-2 upon the vertical adjustment mechanism 125-1 (i.e., similar to the description associated with the incremental visual markings 1000-1 above), to adjust dynamic backrest 105 to a precise height relative to backrest support 110.

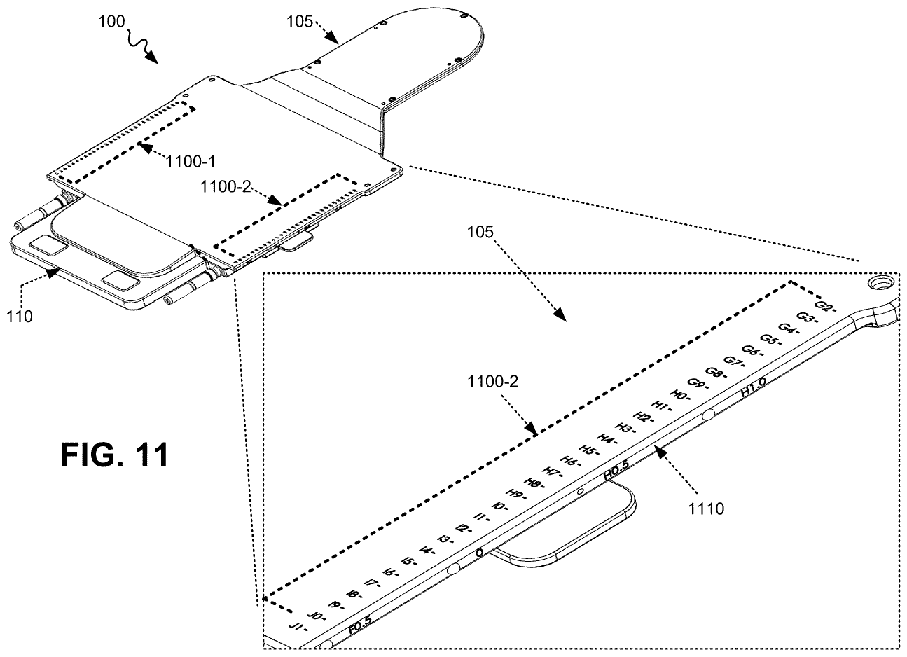

FIG. 11 illustrates an example of incremental visual markings on a front surface of dynamic backrest 105 that may be used for affixing various devices, structures, or accessories to backrest 105 at particular positions. The various devices, structures, or accessories may include, for example, immobilization devices for immobilizing one or more body parts (e.g., the torso) of a patient against backrest 105. Other types of devices, structures, or accessories may be affixed to backrest 105 at positions identified by the incremental visual markings. The devices, structures, or accessories may include various different attachment mechanisms for attaching to the left and/or right edges of dynamic backrest 105, such as, for example, clamps, pins, screws, etc. As shown, incremental visual markings 1100-1 may extend along a left front side of backrest 105, running parallel to the left edge, and similar incremental visual markings 1100-2 may extend along a right front side of backrest 105, running parallel to the right edge.

The incremental visual markings 1100-1 may be used by an operator (not shown) to attach a device, structure, or accessory at a particular location, identified by one of the visual markings, along the left edge of dynamic backrest 105, and, similarly, the incremental visual markings 1100-2 may be used by the operator to attach a device, structure, or accessory at a particular location, identified by another one of the visual markings, along the right edge of dynamic backrest 105. Some of the devices, structures, or accessories may have two ends or sides, with one end/side attaching to the left edge of backrest 105 and another end/side attaching to the right edge of backrest 105, with some part of the devices, structures, or accessories extending either behind or across a portion of the patient's body that is resting against backrest 105. Other devices, structures, or accessories may only attach to a single edge of dynamic backrest 105 and may not extend behind or across the patient's body.

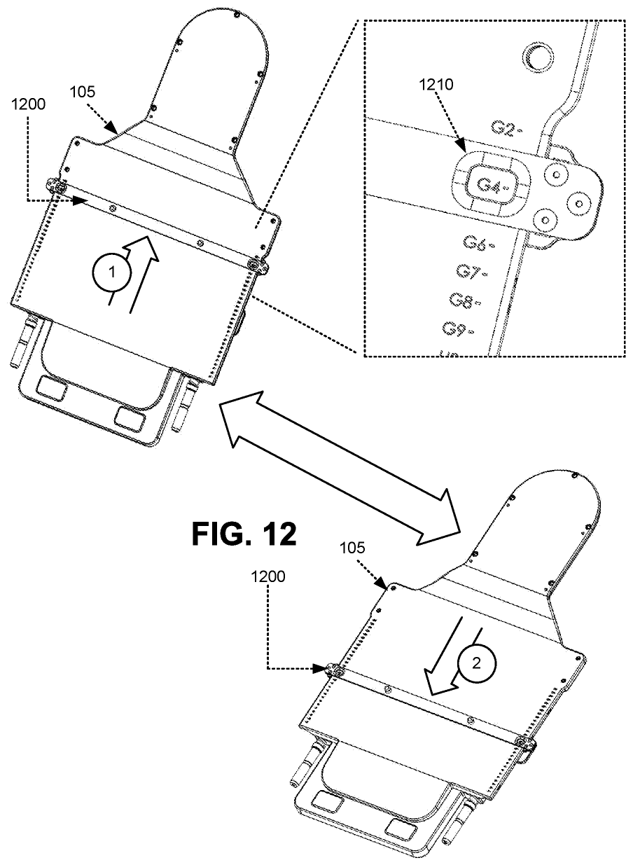

FIG. 12 depicts one example of a device 1200 for attaching to dynamic backrest 105 that has two ends, with one end attaching to the left edge of backrest 105 and a second end attached to the right edge. As shown, device 1200 may be manually moved upwards (identified with a “1”) upon backrest 105 to a particular position 1210 (shown in the upper right corner of FIG. 12). Device 1200 may also be manually moved downwards (identified with a “2”) upon backrest 105 to a second position. As an example, device 1200 may be used in conjunction with mounting or docking points 140 to attach a structure (e.g., a thermoplastic retention material) (not shown) across a patient's head and/or torso to immobilize the patient in a particular position upon backrest 105. As another example, device 1200 may be used for mounting various devices or accessories (not shown) other than patient immobilizing devices or accessories.

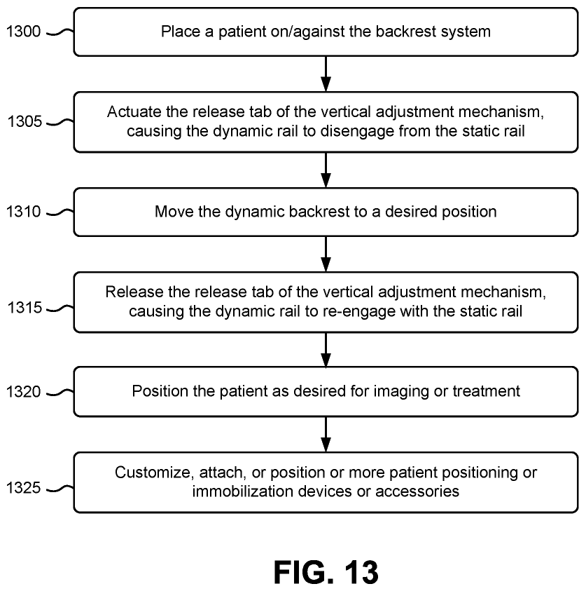

FIG. 13 illustrates an example of a method of use of the backrest system 100. As shown, the method may include the following steps: a) placing a patient on/against the backrest system 100 (block 1300), b) actuating the release tab 400 of the vertical adjustment mechanism 125 such that the release actuator 510 transitions from an engaged state to a disengaged state, causing the teeth of dynamic rail 130 to disengage from the teeth of static rail 120 (block 1305), c) moving the dynamic backrest 105 to a desired position (block 1310), d) releasing the release tab 400 of the vertical adjustment mechanism 125 such that the release actuator 510 transitions from a disengaged state to an engaged state, causing the teeth of dynamic rail 130 to re-engage with the teeth of static rail 120 (block 1315), e) positioning the patient as desired for imaging or treatment (block 1320), and f) customizing, attaching, or positioning one or more patient positioning or immobilization accessories (e.g., device 1200, a mask of thermoplastic retention material, a personalized headrest, a body support cushion) or other types of devices, structures, or accessories upon backrest system 100 (block 1325). Subsequent to steps a) through f) above (i.e., blocks 1300-1325 of FIG. 13), the desired imaging or treatment may be performed. Any of steps a) through f) may be selectively repeated to, for example, re-adjust the position of the dynamic backrest 105, re-position the patient upon/against the backrest system 100, and/or change or re-position a patient positioning or immobilization device or accessory being used.

The foregoing description of implementations provides illustration and description, but is not intended to be exhaustive or to limit the invention to the precise form disclosed. Modifications and variations are possible in light of the above teachings or may be acquired from practice of the invention. For example, while a series of steps have been described with respect to the method of use of backrest system 100 illustrated in FIG. 13, the order of the steps may be varied in other implementations. Moreover, non-dependent steps may be performed in parallel.

Although the invention has been described in detail above, it is expressly understood that it will be apparent to persons skilled in the relevant art that the invention may be modified without departing from the spirit of the invention. Various changes of form, design, or arrangement may be made to the invention without departing from the spirit and scope of the invention. Therefore, the above-mentioned description is to be considered exemplary, rather than limiting, and the true scope of the invention is that defined in the following claims.

All structural and functional equivalents to the elements of the various aspects set forth in this disclosure that are known or later come to be known to those of ordinary skill in the art are expressly incorporated herein by reference and are intended to be encompassed by the claims.

Use of ordinal terms such as “first,” “second,” “third,” etc., does not by itself connote any priority, precedence, or order of one element over another, the temporal order in which acts of a method are performed, the temporal order in which instructions executed by a device are performed, etc., but are used merely as labels to distinguish one element having a certain name from another element having a same name (but for use of the ordinal term) to distinguish the elements.

No element, act, or instruction used in the description of the present application should be construed as critical or essential to the invention unless explicitly described as such. Also, as used herein, the article “a” is intended to include one or more items. Further, the phrase “based on” is intended to mean “based, at least in part, on” unless explicitly stated otherwise.

In the preceding specification, various preferred embodiments have been described with reference to the accompanying drawings. It will, however, be evident that various modifications and changes may be made thereto, and additional embodiments may be implemented, without departing from the broader scope of the invention as set forth in the claims that follow. The specification and drawings are accordingly to be regarded in an illustrative rather than restrictive sense.

What is claimed is:

1. A vertical patient positioning device, comprising:
a backrest support comprising a first vertical planar surface; and
a patient backrest comprising a second vertical planar surface, having mounting points for attaching at least one patient immobilization device, that attaches to the backrest support via a first and a second vertical adjustment assembly that enable the patient backrest to slide relative to the backrest support to adjust a vertical height of the patient backrest.

2. The vertical patient positioning device of claim 1, wherein the first vertical planar surface of the backrest support has a first side and a second side, wherein the first vertical adjustment assembly comprises a first linear rail that attaches to the first side of the backrest support and the second vertical adjustment assembly comprises a second linear rail that attaches to the second side of the backrest support, wherein first components of the first vertical adjustment assembly and second components of the second vertical adjustment assembly releasably engage with the first and second linear rails when sliding relative to the backrest support.

3. The vertical patient positioning device of claim 2, wherein the first components of the vertical adjustment assembly further comprise:
a first dynamic linear rail; and
a first pivoting lever actuator that pivots, based on applied pressure, to cause the first dynamic rail to releasably engage with the first dynamic linear rail.

4. The vertical patient positioning device of claim 3, wherein the second components of the vertical adjustment assembly further comprise:
a second dynamic linear rail; and
a second pivoting lever actuator that pivots, based on applied pressure, to cause the second dynamic linear rail to releasably engage with the second dynamic linear rail.

5. The vertical patient positioning device of claim 1, wherein the backrest support is configured to attach to an upper surface of a patient seat.

6. The vertical patient positioning device of claim 5, wherein the backrest support comprises an insertion member that inserts into a slot of the patient seat and two guide rods that insert into two corresponding holes of the patient seat.

7. The vertical patient positioning device of claim 1, wherein the first vertical adjustment assembly comprises a first linear rail that attaches to the first side of the backrest support and a first dynamic rail coupled to the patient backrest, wherein the first dynamic rail releasably engages with the first linear rail when sliding the patient backrest relative to the backrest support.

8. The vertical patient positioning device of claim 7, wherein the second vertical adjustment assembly comprises a second linear rail that attaches to the second side of the backrest support and a second dynamic rail coupled to the patient backrest, wherein the second dynamic rail releasably engages with the first linear rail when sliding the patient backrest relative to the backrest support.

9. A backrest system for vertical patient positioning, comprising:
a backrest support configured to couple with a patient seat and comprising a first side surface and a second side surface; and
a backrest slidably attached to the first side surface of the backrest support via a first vertical adjustment assembly and to the second side surface of the backrest support via a second vertical adjustment assembly and further comprising a front surface, against which a back of a patient is positioned, wherein the front surface of the backrest includes a plurality of mounting or docking points for attaching at least one patient immobilization device,
wherein the first vertical adjustment assembly comprises:
a first linear rail attached to the first side surface of the backrest support; and
a first vertical adjustment mechanism that releasably engages with the first linear rail to adjust a vertical position of the backrest, and
wherein the second vertical adjustment assembly comprises:
a second linear rail attached to the second side surface of the backrest support; and
a second vertical adjustment mechanism that releasably engages with the second linear rail to adjust the vertical position of the backrest.

10. The backrest system of claim 9, wherein the first vertical adjustment mechanism comprises:
a first pivoting lever actuator; and
a first dynamic rail,
wherein user actuation of the first pivoting lever actuator causes the first dynamic rail to disengage from the first linear rail.

11. The backrest system of claim 10, further comprising:
at least one compression spring configured to apply force to the first dynamic rail to cause the first dynamic rail to engage with the first linear rail.

12. The backrest system of claim 10, further comprising:
a first actuation tab; and
one or more first pivot blocks,
wherein the first pivoting lever actuator pivots about a central axis that extends through the two first pivot blocks, based on user pressure applied to the first actuation tab, and applies force to the first dynamic rail to cause teeth of the first dynamic rail to disengage from teeth of the first linear rail.

13. The backrest system of claim 10, wherein the second vertical adjustment mechanism comprises:
a second pivoting lever actuator;
a second dynamic rail, and
wherein user actuation of the second pivoting lever actuator causes the second dynamic rail to disengage from the second linear rail.

14. The backrest system of claim 13, further comprising:
a second actuation tab; and
one or more second pivot blocks,
wherein the second pivoting lever actuator pivots about a central axis that extends through the two second pivot blocks, based on user pressure applied to the second actuation tab, and applies force to the second dynamic rail to cause teeth of the second dynamic rail to disengage from teeth of the second linear rail.

15. The backrest system of claim 9, wherein a lower portion of the backrest system is configured to attach to the patient seat.

16. A vertical patient positioning device, comprising:
a backrest support comprising a first vertical planar surface;
a patient backrest comprising a second vertical planar surface that includes multiple mounting or docking points for attaching at least one patient immobilization device;
at least one vertical adjustment assembly that interconnects the patient backrest with the backrest support to enable the patient backrest to slide vertically relative to the backrest support,
wherein the at least one vertical adjustment assembly comprises:
a first linear rail that attaches to a first portion of the backrest support,
a first dynamic rail that couples to the patient backrest, and
a first actuation mechanism that causes, based on applied pressure, the first dynamic rail to either engage with the first linear rail to hold the patient backrest at a particular vertical height along the first linear rail relative to the backrest support or disengage from the first linear rail to enable the patient backrest to slide vertically relative to the backrest support, and
wherein the backrest support is configured to attach to a seat assembly.

17. The vertical patient positioning device of claim 16, further comprising:
at least one compression spring configured to apply force to the first dynamic rail to cause the first dynamic rail to engage with the first linear rail.

18. The vertical patient positioning device of claim 16, wherein the first actuation mechanism comprises:
a first actuation tab; and
a first pivoting lever actuator that pivots, based on the pressure applied to the first actuation button, to cause the first dynamic rail to releasably engage with, or disengage from, the first linear rail.

19. The vertical patient positioning device of claim 16, wherein the at least one vertical adjustment assembly comprises a first and a second vertical adjustment assembly, wherein the first vertical adjustment assembly comprises:
the first linear rail,
the first dynamic rail, and
the first actuation mechanism, and
wherein the second vertical adjustment assembly comprises:
a second linear rail that attaches to a second portion of the backrest support,
a second dynamic rail that couples to the patient backrest, and
a second actuation mechanism that causes, based on applied pressure, the second dynamic rail to either engage with the second linear rail to hold the patient backrest at a particular vertical height along the second linear rail relative to the backrest support or disengage from the second linear rail to enable the patient backrest to slide vertically relative to the backrest support.

20. The vertical patient positioning device of claim 19, wherein the second actuation mechanism comprises:
a second actuation tab; and
a second pivoting lever actuator that pivots, based on the pressure applied to the second actuation tab, to cause the second dynamic rail to releasably engage with, or disengage from, the second linear rail.
